(19) United States
(12) Patent Application Publication
(10) Pub. No.: US 2014/0227352 A1
(43) Pub. Date: Aug. 14, 2014

(54) ORAL APHRODISIAC HERBAL COMPOSITION AND CAPSULE

(71) Applicant: EJENDOMSSELSKABET KORSGADE 13, Svenborg (DK)

(72) Inventor: Torben Sogaard-Andersen, Cannes (FR)

(73) Assignee: EJENDOMSSELSKABET KORSGADE 13, Svenborg (DK)

(21) Appl. No.: 13/765,535

(22) Filed: Feb. 12, 2013

(51) Int. Cl. A61K 36/90; A61K 36/185; A61K 45/06; A61K 36/296

(52) U.S. Cl. CPC A61K 36/90; A61K 36/296; A61K 36/185; A61K 45/06
USPC 424/452; 424/725; 424/752

(57) ABSTRACT

A herbal composition comprising extracts or particulate material from the plants Epimedium Grandiflorum, Turnera diffusa var. aphrodisiaca, Ilex paraguariensis, and Sarsaparilla with an admixture of an emulsifying agent and a dispersing agent serves as a female aphrodisiac herbal composition, in particular for women suffering from lack of sexual desire and arousal disorders.

### BACKGROUND

The present invention relates to a female oral aphrodisiac herbal composition comprising extracts or particulate matter from the plants Epimedium Grandiflorum, Turnera diffusa var. aphrodisiaca, Ilex paraguariensis and Sarsaparilla.

During the years focus has been put on the male sexuality, which has been intensively investigated. The treatment of erectile dysfunction has been the subject of many studies and proposed treatments. Numerous male sexual enhancement drugs or pills have been developed and are commercially available today, one of them being VIAGRA®.

Many women experience a loss of sexual vitality at some time in their lives. External factors such as stress and fatigue may contribute to the decline in sexual interest, which often lead to problems in the relationship or even misconduct. However today's emancipated woman attempts to overcome this problem and seeks a solution to improved sexual pleasure and experience; and through the past few years the demand for female sexual desire-promoting means has increased.

A few female products have been developed with the intention to regulate hormone levels, increase female sex drive, increase circulation and relieve anxiety. Such commonly known products are e.g. clitoris-stimulating creams for topical application in relation to or just before sexual intercourse. In addition a few oral compositions including amino acids are offered for sale via the Internet. Intake of large amounts of amino acids may however have a negative influence on the health and should be avoided.

As an alternative, International patent application WO 2006/021930 proposes a herbal composition that has been demonstrated to have significant aphrodisiac effect. Onset, evolving and peak of aphrodisiac effect however do not happen at the best time and to an extent which most women prefer to be able to obtain optimum benefit from the composition and the sexual event.

Accordingly, there is a need for improved products in this area and these are now provided by the present invention.

### SUMMARY OF THE INVENTION

It is a main aspect of the present invention to improve the aphrodisiac effects of the oral aphrodisiac composition disclosed in International patent application WO 2006/021930.

A second aspect of the present invention is to provide an oral aphrodisiac composition having quicker onset and/or quicker evolving of aphrodisiac effect than known herbal compositions that provide females with sexual arousal and desire.

A third aspect of the present invention is to provide a capsule including an aphrodisiac herbal composition for a female's improved pleasurable sexual sensation.

Accordingly, the present invention provides compositions comprising a first extract or particulate matter from a plant selected from the group consisting of Epimedium Grandiflorum, Turnera diffusa var. aphrodisiaca, Ilex paraguariensis and Sarsaparilla, wherein the composition further comprises an admixture of an emulsifying agent and an admixture of a dispersing agent. Advantageously, the composition further comprises a second extract or particulate matter from at least one additional second plant selected from the group consisting of Ginkgo biloba L, Eurycoma longifolia Jack, Rhodiola rosea L, Tribulus terrestris, Lepidum meyenii, Erythroxylum catuaba and Eriosema kraussianum.

In a preferred form, the composition is provided in a orally administratable capsule.

### DESCRIPTION OF THE INVENTION

A detailed description of the main herbal components, which are the basic components of the oral aphrodisiac composition according to the present invention, and which are mentioned in the opening paragraph, is retrievable from International patent application WO 2006/021930.

Briefly summarized Epimedium Grandiflorum is known to support increased libido, improved erectile function, and relief from menopausal discomfort. Turnera diffusa var. aphrodisiaca has been used by the ancient Mayan civilization as an aphrodisiac, and by the Mexican Indians in lovemaking potions. British Herbal Pharmacopoeia cites indications for its use in coital inadequacy. Ilex paraguariensis is not reported as an aphrodisiac but it's effect as a stimulant and of being able to reduce fatigue has been utilized in herbal medicine systems throughout South America and is reported for same in the British Herbal Phamacopoeia (1996). Sarsaparilla or Smilax spp. is reported to contain steroidal saponins, such as sarsaponin or sarsasapogenin, which may mimic the action of some human hormones, e.g. progesterone. Sarsaparilla has been chewed by male Central and Southern Americans for sexual impotence and is used as an ingredient in commercial herbal preparations for libido enhancement. WO 2006/021930 is the first disclosure of combining extracts from the aforementioned four herbal components in an oral aphrodisiac composition.

The present invention attempts to further arouse sexual desire of females far beyond what was achievable by the compositions known from International patent application no. WO 2006/021930 in order to induce further and more controlled pleasurable sensation of females.

The novel and unique whereby the above and other aspects are achieved according to the present inventions consists in the fact that the composition further comprises an admixture of an emulsifying agent and an admixture of a dispersing agent. The admixed emulsifying agent and the dispersing agent act together with the herbal components to induce a very significant positive influence on sexual arousal far beyond expectations. Tests have revealed that users experience more sex initiative, more satisfying sexual intercourse, increased sensitivity and blood filling of the clitoris, increased and more frequent lubrication, and in general a more satisfying sex life.

The admixed emulsifying agent advantageously reduces surface-activity of at least the extracts or particulate matter from the plants, i.e. the herbal components. The dispersing agent is admixed to disperse the individual components of the herbal composition not only in the bulk product, i.e. in the doses of herbal composition before ingestion, but also when ingested. The dispersing agent provides the herbal components with a large surface to be exposed for the influence and action of other components, including the emulsifying agent that serves an important function in contributing to quickly extract high levels of aphrodisiac active substances from the extracts or particulate plant matter of the herbal composition so that these active substances can pass further into the blood circulation at high speed and rate at minimum delay after oral intake of the herbal composition.

A preferred admixed emulsifying agent may include lecithin. The emulsifying agent preferably constitutes about 1-25% by weight of the total composition, more preferred between 5-15% by weight of the total composition, and most preferred about 12% by weight of the total composition. Suitable surface-active, hydrophilic and lipophilic lecithins that allow the herbal components to mix easily into a stable composition wherein separation of components is almost completely avoided are obtainable from LECICO GmbH, Theodorstrasse 88, Westend Village/Haus 10, 22761 Hamburg. A suitable lecithin product is LECICO Sun 400, which is a sunflower lecithin.

A preferred admixed dispersing agent may include bicarbonate, preferably sodium bicarbonate. The dispersing agent preferably constitutes about 1-50% by weight of the total composition, more preferred between 15-35% by weight of the total composition, and most preferred about 20% by weight of the total composition. Upon contact with gastric juice bicarbonate develops carbon dioxide. The gas pressure from the carbon dioxide causes the constituents of the composition to get apart and disperse. The overall surface of the herbal composition is in this manner instantaneously made very large and accessible for the emulsifying agent to take action to extract the much desired active substances from the plant ingredients.

Admixture of an emulsifying agent together with a dispersing agent significantly serves to increases arousal effects incident of the known herbal composition disclosed in WO 2006/021930.

Effective amounts per 48 hours of extracts or particulate matter to be combined with amounts of emulsifying agent and dispersing agent in accordance with the present invention are
between 100-300 mg Epimedium Grandiflorum, preferably between 180-210 mg,
between 40-80 mg Turnera diffusa var. aphrodisiaca, preferably 55-70 mg,
between 40-80 mg Ilex paraguariensis, preferably preferably 55-70 mg, and
between 3-8 mg Sarsaparilla, preferably between 5-7 mg.

Advantageously, the composition may further comprise an extract or particulate material from at least one additional second plant as described in International patent application WO 2006/021930 and to which references are made for a detailed discussion of suitable second plants.

The second plant can be e.g. be Ginkgo biloba L. extracts of which has been found to make women more sexually responsive. Another second plant can be Eurycoma longifolia Jack, the root of which has been utilized as aphrodisiac for males. Yet another second plant can be Rhodiola rosea L., which has been used in women suffering from amenorrhea. A fourth second plant can be Tribulus terrestris, which has long been used in the traditional Chinese and Indian systems of medicine to improve sexual functions in man. A fifth second plant can be Lepidum meyenii, which traditionally has been used in the Andes to enhance fertility in humans and animals. Optionally extracts or particulate material from Erythroxylum catuaba can also be added. A bark decoction of Erythroxylum catuaba has been reported used for sexual impotency and weakness by the male Brazilian Indians. As a variation extract or particulate material from the rootstock of Eriosema kraussianum may be included in the composition.

Preferred amounts of extract or particulate material from the at least one second plant is between 10-30 mg of Ginkgo biloba L, preferably from the leaves of Ginkgo biloba L, between 10-30 mg of Eurycoma longifolia Jack, preferably from the root of Eurycoma longifolia Jack, between 30-40 mg of Rhodiola roesea L., preferably from the root of Rhodiola roesea L., between 10-50 mg of Tribulus terrestris, preferably from the fruits of Tribulus terrestris and between 10-50 mg of Lepidum meyenii, preferably from the root of Lepidum meyenii.

When extract or particulate material from Erythroxylum catuaba is included in the composition it is preferred to include 400-1000 mg extract or particulate material from the bark of Erythroxylum catuaba. When extract or particulate material from Eriosema kraussianum is included in the composition it is preferred to include 300-400 mg extract or particulate material from the rootstock of Eriosema kraussianum.

At least one additive, such as a filling agent and/or a flavouring agent and/or a vitamin and/or a mineral can also be added if preferred.

A preferred filling agent is lactose or starch, both of which commonly are used as bulk material in medicaments for oral use. The filling agent serves for providing the composition with a suitable appearance. Any vitamin can be included in the composition within the scope of the present invention if desired. Preferred minerals are included as their salts, e.g. calcium or magnesium. Some herbal extracts have an unpleasant taste, which can be camouflaged by means of a flavouring agent to improved comfort for the woman during oral intake.

The herbal composition is conveniently mixed together with the emulsifying agent and the dispersing agent and encapsulated using techniques known to the person skilled in the art. Mixing of all components can e.g. include crushing or grounding herbal ingredients in the presence of an admixture of the emulsifying agent and the dispersing agent. The crushed mixtures is then compressed and filled into capsules. As an example, a capsule may have a basic content of the active herbal ingredients in a total amount of 300-400 mg, e.g. 340 mg. A recommended prescription is three of these capsules taken orally at least 1 hour before sexual activity. The advantage of sexual desire will sustain during approximately 48 hours after oral ingestion. No special reasons are needed before start of treatment. In contrast to the few known oral compositions, the composition according to the present invention is a short acute treatment. No permanent inexpedient increase in the oestrogenic level is released and consequently the women's overall hormone balance remains substantially unaffected in the long term. No reports have been found on severe toxicity and the inventor of the present invention considers the present improved aphrodisiac herbal composition as extremely safe products.

### COMPARATIVE EXAMPLE

### Method:

48 women were recruited from an urban population in Draguignan, France. Each was interviewed by a clinician and invited to participate in a study to evaluate the effectiveness of the composition of herbs defined in Table 1 below. No other additives are included. During the interview each woman was provided with either the composition according to the present invention (Group A; n=24) or the composition defined in International patent application WO 2006/021930 (Group B; n=24) and asked to take the respective composition in connection with sexual activity over the next 10 days.

TABLE 1

| | Amount in mg | Amount in mg |
|---|---|---|
| Component | Group A | Group B |
| Epimedium Grandiflorum | 200 | 200 |
| Turnera diffusa var. aphrodisiaca | 67 | 67 |
| Ilex paraguariensis | 67 | 67 |
| Sarsaparilla | 6 | 6 |
| Sodium hydrogencarbonate | 100 | 0 |
| Lecico 400 | 60 | 0 |
| Other additives | 0 | 188 |

Neither the clinician nor the participants knew, during the interview, whether they had been provided the new composition or the old one. The test lasted 48 hours once the participant's had taken the composition. At the conclusion of the test, each participant was re-interviewed by the clinician and asked to complete a questionnaire about their sexual experience during the trial.

### Main Outcome Measure:

The primary outcome measure was the participant's response to a yes/no questionnaire designed to obtain information in relation to their sexual excitement whilst using the handed-over respective composition.

### Results:

| | Percentage of | Percentage of |
|---|---|---|
| Question | group A (%) | group B (%) |
| Have you taken more initiative | 88 | 69 |
| regarding sex during the 48 hours? | | |
| Has sexual intercourse in general | 96 | 51 |
| been more satisfying? | | |
| Have you noticed increased | 89 | 12 |
| sensitivity of the clitoris? | | |
| Have you noticed increased | 68 | 12 |
| blood filling of the clitoris? | | |
| Have you noticed increased | 98 | 16 |
| lubrication during the test? | | |
| Have you noticed more | 88 | 15 |
| frequent lubrication? | | |
| Have orgasms in general | 90 | 19 |
| been more satisfying? | | |
| Has your sex life in general been | 98 | 18 |
| more satisfying in the 48 hours? | | |

Most women of Group A further expressed unasked that they had much faster onset of sexual stimulation when using the composition according to the present invention than when using the composition known from International patent application no. WO 2006/021930. Some women even meant that onset took place double as fast.

### CONCLUSIONS

The questionnaire scores demonstrate a highly significant difference between the two groups. Distinct side effects were not proven. The test shows that when an emulsifying agent and a dispensing agent is added to the herbal composition a highly significant positive influence on sexual arousal and response is achieved.

It has been demonstrated that the oral aphrodisiac herbal compositions including a emulsifying agent and a dispersing agent in accordance with the present invention induce an improved widespread feeling of vigour, sexual attraction and increased sexual pleasure faster than when using known herbal compositions. Female test persons expired an increased blood circulation to the clitoris and increased stamina and sex drive. The test women of Group A having the composition according to the present invention in general had a sex life that was more satisfying during the 48 hours.

The treatment of any female animal other than the human being is also considered within the scope of the present invention.

What is claimed is:

1. A female oral aphrodisiac herbal composition comprising a first extract or particulate matter from a plant selected from the group consisting of Epimedium Grandiflorum, Turnera diffusa var. aphrodisiaca, Ilex paraguariensis and Sarsaparilla, wherein the composition further comprises an admixture of an emulsifying agent and an admixture of a dispersing agent.

2. The composition according to claim 1, wherein the admixed emulsifying agent includes lecithin.

3. The composition according to claim 1, wherein the emulsifying agent constitutes between 1-25% by weight of the total composition.

4. The composition according to claim 1, wherein the emulsifying agent constitutes between 5-15% by weight of the total composition.

5. The composition according to claim 1, wherein the emulsifying agent constitutes about 12% by weight of the total composition.

6. The composition according to claim 1, wherein the admixed dispersing agent includes bicarbonate.

7. The composition according to claim 6, wherein the admixed dispersing agent is sodium bicarbonate.

8. The composition according to claim 1, wherein the dispersing agent constitutes between 1-50% by weight of the total composition.

9. The composition according to claim 1, wherein the dispersing agent constitutes between 15-35% by weight of the total composition.

10. The composition according to claim 1, wherein the dispersing agent constitutes about 20% by weight of the total composition.

11. The composition according to claim 1, wherein effective amounts (per 48 hours) of the first extract or particulate matter are:
between 100-300 mg or between 180-210 mg Epimedium Grandiflorum,
between 40-80 mg or between 55-70 mg Turnera diffusa var. aphrodisiaca,
between 40-80 mg or between 55-70 mg Ilex paraguariensis, and
between 3-8 mg or between 5-7 mg Sarsaparilla.

12. The composition according to claim 1, wherein the composition further comprises an extract or a particulate matter from at least one additional second plant selected from the group consisting of Ginkgo biloba L, Eurycoma longifolia Jack, Rhodiola rosea L, Tribulus terrestris, Lepidum meyenii, Erythroxylum catuaba and Eriosema kraussianum.

13. The composition according to claim 12, wherein the effective amounts (per 48 hours) of the second extract or particulate material of the at least one additional second plant are between 50-70 mg of Ginkgo biloba L, between 10-30 mg of Eurycoma longifolia Jack, between 30-40 mg of Rhodiola roesea L., between 10-50 mg of Tribulus terrestris, between 10-50 mg of Lepidum meyenii, between 400-1000 mg of Erythroxylum catuaba, and between 300-400 mg of Eriosema kraussianum.

14. The composition according to claim 1, which further comprises at least one additive, said additive being a filling agent, a flavouring agent, a vitamin or a mineral.

15. A female oral aphrodisiac herbal composition comprising:
an effective amount of a first extract or particulate matter from a plant selected from the group consisting of Epimedium Grandiflorum, Turnera diffusa var. aphrodisiaca, Ilex paraguariensis and Sarsaparilla,
an admixed emulsifying agent that includes lecithin and that constitutes between 1-25% by weight of the total composition;
an admixture of a dispersing agent that includes bicarbonate and that constitutes between 15-35% by weight of the total composition; and
an effective amount of a second extract or a particulate matter from at least one additional second plant selected from the group consisting of Ginkgo biloba L, Eurycoma longifolia Jack, Rhodiola rosea L, Tribulus terrestris, Lepidum meyenii, Erythroxylum catuaba and Eriosema kraussianum;
wherein the effective amounts (per 48 hours) of the first extract or particulate matter are:
between 100-300 mg or between 180-210 mg Epimedium Grandiflorum,
between 40-80 mg or between 55-70 mg Turnera diffusa var. aphrodisiaca,
between 40-80 mg or between 55-70 mg Ilex paraguariensis, and
between 3-8 mg or between 5-7 mg Sarsaparilla; and
wherein the effective amounts (per 48 hours) of the second extract or particulate matter are:
between 50-70 mg of Ginkgo biloba L,
between 10-30 mg of Eurycoma longifolia Jack,
between 30-40 mg of Rhodiola roesea L.,
between 10-50 mg of Tribulus terrestris,
between 10-50 mg of Lepidum meyenii,
between 400-1000 mg of Erythroxylum catuaba, and
between 300-400 mg of Eriosema kraussianum.

16. The composition of claim 15 which consists essentially of the recited ingredients.

17. The composition of claim 15 which consists of the recited ingredients and optionally contains at least one additive, said additive being a filling agent, a flavouring agent, a vitamin or a mineral.

18. A capsule that includes therein the composition according to claim 1.

19. A capsule that includes therein the composition according to claim 15.

20. A capsule that includes therein the composition according to claim 17.
